(19) United States
(12) Patent Application Publication
(10) Pub. No.: US 2003/0097180 A1
(43) Pub. Date: May 22, 2003

(54) Joint prosthesis

(76) Inventors: Pertti Tormala, Tampere (FI); Mauri Lehtimaki, Kangasala (FI); Matti Lehto, Tampere (FI); Minna Kellomaki, Tampere (FI); Pirjo Honkanen, Ylojarvi (FI)

Correspondence Address: KENYON & KENYON, 1500 K STREET, N.W., SUITE 700, WASHINGTON, DC, 20005, US

(21) Appl. No.: 09/988,777

(22) Filed: Nov. 20, 2001

(51) Int. Cl. A61F 2/28

(52) U.S. Cl. 623/13.18; 623/21.15; 623/23.57; 623/23.59; 606/77

(57) ABSTRACT

The present invention provides a joint prosthesis system for the joining of two bones. The bioabsorbable joint prosthesis system includes at least one bioabsorbable spacer and at least one connector adapted to be fixedly attached to the two bones, at least a portion of the connector being in contact with the spacer to prevent lateral movement of the spacer. The present invention also includes embodiments drawn to methods of using the joint prosthesis system. In an embodiment of the present invention, the method includes interposing at least one bioabsorbable spacer between the surface of the bones to be joined and connecting the bones with at least one connector such that at least a part of the connector contacts the bioabsorbable spacer.

### FIELD OF THE INVENTION

The present invention relates to the field of implantable joint prostheses. Specifically, this invention relates to a joint prosthesis intended to be mounted between two bones to be joined together, wherein the joint prosthesis comprises a spacer intended to be placed between the joint surfaces of the bones to be joined, and which is manufactured of biodegradable polymer, copolymer, polymer mixture and/or composite, and connectors which may be constructed of the patient's own fibrous or soft tissue and which contact the spacer and help maintain its position between the bones to be joined, thereby forming a stable joint replacement.

### BACKGROUND OF THE INVENTION

Biohybrid, bioreplaceable joint prostheses are a new concept in joint surgery; thus far, prosthetic materials have been limited to biostable materials, particularly for joints between small bones in the hands and feet. The manufacturing of a joint prosthesis from synthetic, elastic biostable (non-degradable) plastics is well known in the art. Artificial biostable joint prostheses are commercially available, for example, from Dow Corning, S.A., Valbourne Cedex, France, under the trade name Silastic®. Such an artificial joint is typically composed of a spacer, which is positioned between the bones to be joined, and two elongated anchors, which are anchored in the bones to be joined.

However, there are a number of drawbacks to the use of the joint prostheses manufactured of biostable polymers, polymer mixtures and elastomers. For instances, when a biostable prosthesis is used the operated limb can only withstand a set amount of strain following the operation. Thus, a permanent strain limit for the operated limb has to be set, which may limit the post-operation activities of the patient. For example, when a Silastic® joint prosthesis is used to replace a finger joint, the operated hand may be strained only with a burden of 5 kg. Overstraining may lead to loosening, breaking or erosion of the implant, which forms the joint prosthesis.

Furthermore, the erosion and/or corrosion of a biostable joint prosthesis may cause loose particles to be released from the joint prosthesis, which may cause a chronic inflammation reaction, e.g., a synovitis, and/or osteolytic changes in the bone. Further, the inflammation may cause tumefaction and pain in the joint, possibly requiring the removal of the joint prosthesis.

There have been attempts by inventors to address the problems associated with biostable implants by designing implants comprised of resorbable materials that were intended to reconstruct a joint. For example, a device that attempts to address the problem with interposition devices for the repair of small joints is disclosed in Berman (U.S. Pat. No. 6,017,366), wherein the implantable device comprises a structural article having a non-resorbable core provided with a resorbable shell. This type of device still suffers from the same disadvantage of the previous devices in that the non-resorbable core or elastic material may cause long-term problems as other non-resorbable joint prostheses. More recently, there have been attempts to make devices that are comprised entirely of resorbable materials. For example, Lehto et al. (U.S. Pat. No. 6,007,580) discloses a two piece bioresorbable joint prosthesis that is comprised of a porous spacer part and proximal and distal fixation parts, which are fixed to the bones to be joined. However, the use of fixation parts that are made of synthetic bioabsorbable material forms an auxiliary bioburden in addition to the porous joint spacer. Additionally, Tormala et al. (U.S. Pat. No. 6,113,640) describes a prosthesis for implantation comprised of a porous joint spacer made by wrapping a bioabsorbable fabric into a cylindrical body and a bioabsorbable fixation part, wherein the fixation part is capable of fixing said cylindrical body to a bone. A typical fixation part of this invention can be, e.g., a rod, bar, screw, a cloth or a loop of suture. The fixation part of U.S. Pat. No. 6,113,640 also can be constructed of the patient's own fibrous tissue, such as tendon or ligament tissues. However, these devices require the fixation part(s) to penetrate the joint spacer, thus creating a risk of damage of the joint spacer or fixation parts.

Various other implant devices made from resorbable material have been described. These consist primarily of devices that include fixation parts such as plates, pins, and screws to fix the joint spacer in the joint cavity to the bone. These devices are designed to hold the adjacent ends of the adjacent bones of a particular joint in appropriate relationship while accommodating tensile loads, thus preventing further separation of the adjacent bones during use of the joint.

A need exists for bioreplaceable (bioresorbable) joint prosthesis device, which is constructed of a minimal amount of foreign, synthetic material and which is fixed into a joint cavity with a minimal risk of damaging either the joint spacer or fixation part. The present invention relates to a bioreplaceable joint prosthesis that provides improved performance in comparison to prior devices.

### SUMMARY OF THE INVENTION

The present invention provides a bioabsorbable joint prosthesis system for joining two bones. The bioabsorbable joint prosthesis system, which can create a new, functional joint in situ, comprises at least one bioabsorbable spacer and at least one connector.

The present invention also includes embodiments drawn to methods of using the bioabsorbable joint prosthesis. In an embodiment of the present invention, the method includes interposing at least one bioabsorbable spacer between the surface of the bones to be joined and connecting the bones with at least one connector such that at least a part of the connector contacts the bioabsorbable spacer.

### BRIEF DESCRIPTION OF THE DRAWINGS

FIG. 1 illustrates a sagittal sidewise cross-section of a metacarpophalangeal joint of a thumb treated with an embodiment of the present invention, where the joint surfaces have been removed and a cylindrical joint spacer is located between the ends of the bone and maintained in its place with two collateral ligaments, which touch the joint spacer.

FIG. 2 illustrates a prosthesis comprised of two cylindrical bodies and two connectors (in this embodiment, collateral ligaments), wherein a cavity is formed between the two cylindrical bodies.

FIG. 3 illustrates embodiments of joint spacers (scaffolds) used in MCP joints as part of the present invention.

FIG. 4 is a graph illustrating preoperative and postoperative mean active flexion for patients treated with embodiments of the present invention.

FIG. 5 is a graph illustrating preoperative and postoperative mean active extension lag for patients treated with embodiments of the present invention.

FIG. 6 is a graph illustrating preoperative and postoperative mean ulnar deviation for patients treated with embodiments of the present invention.

FIG. 7 is a graph illustrating preoperative and postoperative mean volar subluxation for patients treated with embodiments of the present invention.

FIG. 8 is a graph illustrating preoperative and postoperative range of motion for patients treated with embodiments of the present invention.

FIG. 9 is a graph illustrating preoperative and postoperative pain of patients for patients treated with embodiments of the present invention.

### DETAILED DESCRIPTION OF THE PREFERRED EMBODIMENTS

The present invention provides an implantable biohybrid bioreplaceable (i.e., bioabsorbable) joint prosthesis, having improved properties and functional characteristics. The biohybrid bioreplaceable joint prosthesis of the present invention may include a cylindrical, porous joint spacer and at least one connector, which is comprised of the patient's tissue. The biohybrid bioreplaceable joint prosthesis of the present invention can be used, for instance, in a joint cavity in hands and feet to regenerate the joint. Because the connector maintains the position of the joint spacer in the joint cavity by contacting the outer surface of the spacer, there is no need for the connector to penetrate the joint spacer, thus eliminating the risk of damage of the joint spacer or connector.

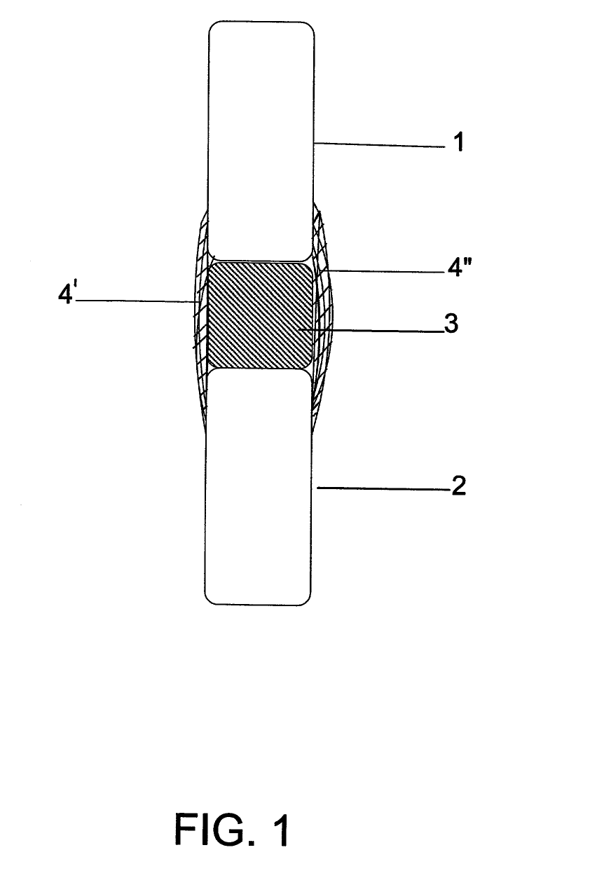

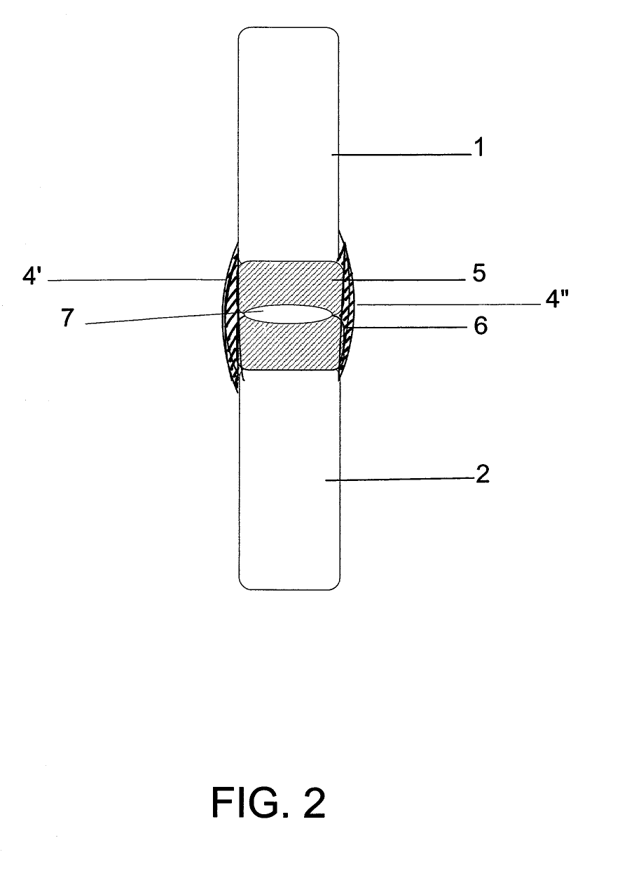

FIG. 1 shows a preferred embodiment of the joint prosthesis of the present invention, connecting bones 1 and 2, comprising a cylindrical body 3 and in this case two connectors 4′ and 4″. In FIG. 2, the surfaces of cylindrical bodies 5 and 6 that are in contact with the bones 1 and 2 can be coated with a bone growth promoting substance, such as bone morphogenic proteins (BMP) or with another BMP releasing bioabsorbable polymer or with a bioactive ceramic material to facilitate ossification of each cylindrical body 5 and 6 into the corresponding bone.

In certain preferred embodiments of the present invention, the cylindrical, porous joint spacer can be manufactured using fibers or a cylinder of a biodegradable polymer, co-polymer, polymer mixture or composition, or by combining various biodegradable polymer substances. In the medical, technical and patent literature, a multitude of bioabsorbable (biodegradable) polymers have been identified that are suitable as raw materials for making a joint spacer in accordance with the present invention. These include, for example, bioabsorbable aliphatic polyesters (cf., e.g., Vainionp\ddot{aa}, S., Rokkanen, P., and Törmälä, P. in Progr. Polym.Sci., 14 (1989) pp. 679-716; U.S. Pat. Nos. 4,743,257, 5,084,051, 4,968,317; EPO Application No. 0423155; and PCT application No. PCT/FI93/00014); and polyester amides, polyorthoesters, polyanhydrides and polyphosphazenes (cf., e.g., C. T. Laurensin et al., J. Biomed Mater. Res. 27 (1993), pp. 963-973), the disclosures of which are incorporated herein by reference, in their entirety.

The cylindrical porous joint spacer of the present invention can have a structure as disclosed in the prior art, see, e.g., U.S. Pat. No. 6,007,580, U.S. Pat. No. 6,113,640, or U.S. Pat. No. 6,017,366, the disclosures of which are incorporated herein by reference, in their entirety. In various embodiments of the present invention, the mechanical properties, porosity and degradation behaviour of the spacer can be varied by applying methods described in the prior art incorporated above.

When located in a joint cavity, the joint spacer of the present invention will be covered and/or filled relatively rapidly with connective tissue. This process will be facilitated by the connectors (e.g., the balanced collateral ligament(s) and/or the joint capsule) that prevent horizontal movements of the joint spacer. During the bioabsorption process, the joint spacer is replaced by a biological, fibrous tissue and simultaneously the balanced collateral ligaments or joint capsule heal. As a result, a new, biological, elastic fibrous tissue joint is obtained, which allows movement of the joint bones by the surrounding muscles. As the new joint is formed during the degradation process of the joint spacer, no foreign particles are released that are chronically harmful to the patient's system, as can be the case with biostable joint prostheses. Additionally, because the connectors do not penetrate the joint spacer, there is no such risk of damage of the joint spacer or connectors, as in the case of prior art bioabsorbable prostheses, where fixation parts penetrates the joint spacer.

To permit tissue growth within the joint spacer after its implantation, the cylindrical body of the present invention is preferably and advantageously porous, with the pore size varying between, e.g., 50 μm and 1000 μm. The pore size of the cylindrical body can be varied, as illustrated in the prior art, in accordance with the desired mechanical strength of the prosthesis and distance between the bones to be joined.

In various embodiments of the present invention, the stiffness, flexibility, surface quality and porosity of the cylindrical body, which is used to manufacture the spacer body, can be controlled by annealing the cylindrical body at elevated temperatures (typically, at a temperature T>T_{g}, where T_{g} is the glass transition temperature the polymer component of the cylindrical body). This procedure is optimally performed in a suitable mold and under mechanical pressure. Annealing and the simultaneous mechanical pressure make the cylindrical body stiffer and, if the treatment is done in a mold, the form of the cylindrical body can be changed permanently, e.g., the circular geometry of the cylindrical body can be flattened or its even surfaces can be made curved.

In other preferred embodiments, the joint spacer of the present invention may also include various additives to facilitate the processability of the material (for example stabilizers, antioxidants, or softening agents) or to change its properties (for example softening agents or ceramic chemicals in powder form or bioabsorbable ceramic fibers, such as bioactive glass fibers) or to facilitate its use (e.g., colouring agents).

According to one advantageous embodiment of the invention, the joint spacer contains a bioactive agent or agents, such as antibiotics, chemotherapeutic agents, agents accelerating wound healing, agents inducing the forming of cartilage collagen or chondrocytes, growth hormones, anticoagulant (such as heparin), etc. Bioactive mediums of this type are particularly advantageous in clinical use, because, in addition to the mechanical effect, they have biochemical effects (for example, accelerating the growth of fibrous and/or cartilage tissue, and/or bone tissue), medical and other beneficial effects in human tissues.

In another advantageous embodiment of the present invention shown in FIG. 2, the joint spacer comprises two cylindrical bodies 5 and 6, which can be located parallel to one another in the joint cavity. In such a configuration, a vertical cavity 7 is left between the cylindrical bodies, simulating the synovial joint cavity. When the patient moves the joint following such an implantation, the cylindrical bodies glide in relation to each other and the synovial cavity-like space can remain inside the growing fibrous joint.

In another advantageous embodiment of the invention, the contacting surfaces of cylindrical bodies 5 and 6, which contacting surfaces form the walls of the cavity 7 in FIG. 2, can be coated with hyaline cartilage cells and/or with growth factors or other bioactive substances (or with another bioabsorbable polymer that releases growth factors), promoting the growth of hyaline cartilage or the formation of a cartilage layer on the cavity surfaces of the growing joint. In another preferred embodiment, the surfaces of the cylindrical bodies 5 and 6 that are in contact with the bones 1 and 2 can be coated with a bone growth promoting substance, such as bone morphogenic proteins (BMP), or with another BMP releasing bioabsorbable polymer or with a bioactive ceramic material to facilitate ossification of each cylindrical body 5 and 6 into the corresponding bone.

In yet another embodiment of the present invention, a flat hole or circular fissure located inside the cylindrical body simulates a synovial cavity.

Connectors of the joint prosthesis in accordance with the present invention work in conjunction with the joint spacer to form a flexible joint prosthesis. The connectors help maintain the position of the joint spacer between the bones to be joined, wherein by means of muscular power it is also possible to bend the bones to be joined in relation to each other. As a result, a new, biological, elastic fibrous tissue joint is obtained, which allows movement of the joint bones by the surrounding muscles. As the new joint is formed, during the degradation process of the joint spacer no foreign particles are released that are chronically harmful to the patient's wellbeing, as can be the case with the so-called biostable prior art joint prostheses. Thus, the joint prosthesis of the present invention entirely eliminates the risks of such chronic complications caused by loose foreign particles.

The joint prosthesis of the present invention performs surprisingly well after implantation, whether one or both of the bones to be joined have had the joint surface removed. In one embodiment, if the joint surface is removed only from one bone and not removed from the other bone, one surface of the implanted cylindrical body can be made concave and the other surface planar. In an alternative embodiment, both surfaces of the cylindrical body can be made concave to fit the convex joint surfaces of the two bones to be joined.

The performance of the present invention is further illustrated with reference to the following non-limiting examples.

### EXAMPLE 1

Manufacture of the porous scaffold (joint spacer) using a biodegradeable co-polymer of an L-lactic acid and D-lactic acid:

As raw material for the spacer part, an L and D-lactic acid co-polymer with L,D-monomer ratio 96 to 4 (P(L/D)LA 96/4, PLA96) was used. The polymer was medical grade, highly purified material.

PLA 96 (Purac biochem bv, Gorinchem, The Netherlands) was melt-spun to 4-ply multifilaments, following the method described by M. Kellomäki, et al., in “In vivo degradation of composite membrane of P(e-CL/L-LA) 50/50 film and P(L/D)LA 96/4 mesh” Materials for Medical Engineering: Euromat Volume 2, edited H. Stallforth and P. Revell. Wiley-VCH, Weinheim, Germany, 2000; 2:73-79, incorporated herein by reference. The yarn was knitted to a tubular mesh form using a 20-needle cylinder in a tubular single jersey knitting machine (Textilmaschinenfabrik Harry Lucas GmbH & Co KG, Neumünster, Germany). The knitted tube was rolled to form cylindrical scaffolds, which were γ-sterilized prior to use. Scaffolds have open porosity throughout the structure, formed by mesh loops and by layers of the mesh.

The yarns were tensile tested at a crosshead speed of 30 mm min⁻¹ using an Instron 4411 materials testing machine (Instron plc, High Wycombe, England). Pneumatic grips were used, and gauge length was 100 mm. Initial tensile results were measured on dry specimens, and, after in vitro hydrolysis, wet specimens were tested. Mean and standard deviations of stress and strain at maximum load were calculated.

Diameters of the melt-spun PLA96 fibers (single filaments of the yarns) varied between 70-100 μm depending on the produced and used batch. The initial tensile strength of 4-ply fibers was between 450-600 MPa with a Young's modulus of 6.5-8.5 GPa. Variation at this scale did not influence the properties of the scaffolds. Filaments retained 50% of their strength for at least 13 weeks in vitro. X-ray results and post-operation joint functionality indicate that this strength retention is sufficient to allow the spacer to retain its size and shape in situ long enough for tissue ingrowth and maturation.

The strength retention of the filaments in vitro can be used for quality control of the scaffolds and the presented values can be used as acceptable limits.

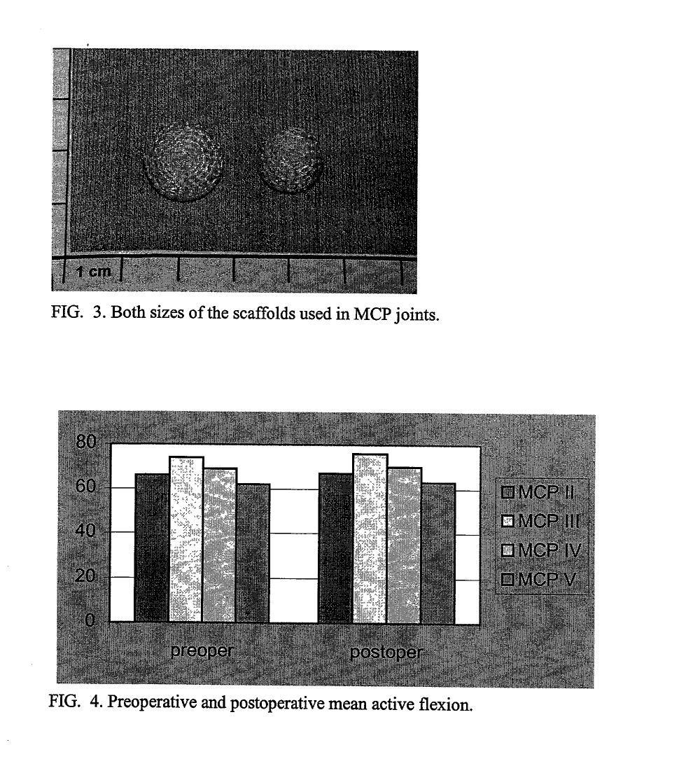

FIG. 3 shows embodiments of a design for highly porous cylindrical scaffolds made of PLA96 filaments for MCP joint prostheses. Slightly shaped cylinders were found to fit well into the joint space between metacarpus and phalanx. Scaffolds that are too soft or that degrade too rapidly may cause a collapse of the joint space as well as restricted and twisted movements of the fingers. On the other hand, scaffolds that are too rigid may prevent postoperative rehabilitation. It was found that the scaffolds of FIG. 3 have the proper balance of mechanical properties to be useful in effective joint repair.

### EXAMPLE 2

Bioreplaceable joint prostheses manufactured according to example 1 were used as artificial joints to replace knuckle joints.

The joint prostheses were implanted into metacarpophalangeal (MCP) joints in patients with rheumatoid arthritis. The porous scaffolds (joint spacers) were held in place within the joint by the contact between the surfaces of the scaffolds and the connectors which were comprised of the patients' own fibrous tissue or soft tissue.

The soft tissue balancing in the bioreplaceable implant arthroplasties of this example applied the following principles. Stability of the joint and prevention of ulnar drift and volar subluxation deformities was maintained through balancing of the soft tissues, see, e.g., Chung et al., in “Patient Outcomes Following Swanson Silastic Metacarpophalangeal Joint Arthroplasty in the Rheumatoid Hand: A Systematic Overview”, J Rheumatol. 2000; 27:1395-1402. The quantity and quality of soft tissue balancing required during the operations was determined by the grade and type of deformity. When ulnar deviation existed, ulnar collateral ligaments were practically always released. The ulnar intrinsic muscle contractures were evaluated and released in stages. Release may include both the bony and winged portions. The abductor digiti minimi tendon of the fifth finger was always released. The tightening of radial collateral ligaments can be performed by duplicating or re-fixing ligament more proximally through the bone canals. If the radial collateral was inadequate, as often in cases with advanced destruction, a cross-intrinsic transfer was utilised to augment the radial support structures. Adequate correction of volar subluxation usually needed the discision of the volar plate. If correction of volar subluxation was difficult, stabilization with an extensor tendon tenodesis was performed. At the end, the extensor tendon was centralised from the typical ulnar dislocation.

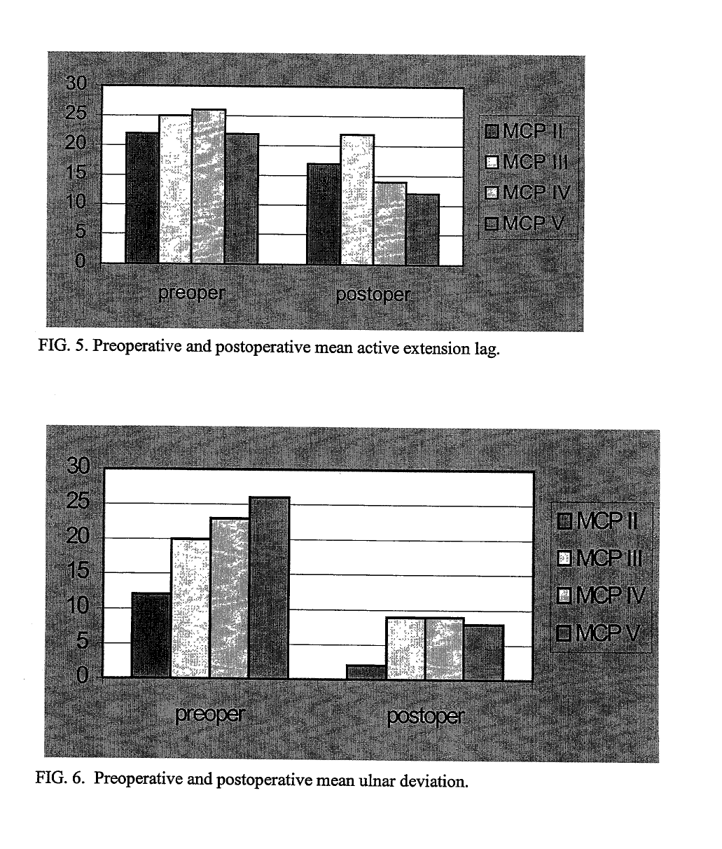

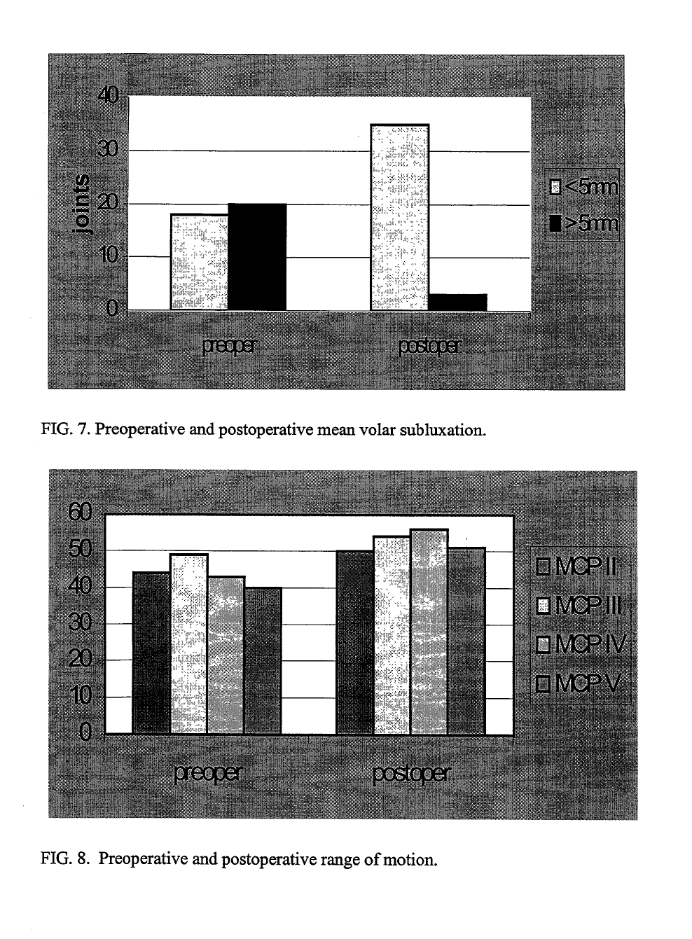

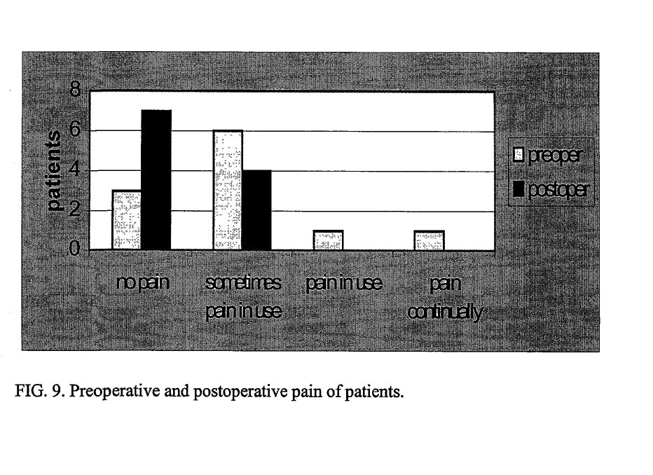

Observed mean results are shown in FIG. 4 for the mean active flexion and in FIG. 5 for the mean active extension. As shown in FIG. 6, the ulnar deviation was preoperatively 20° and 4° postoperatively. In FIG. 7, volar subluxation was observed in all joints preoperatively and in eight (21%) joints postoperatively. FIG. 8 shows an improvement in the range of motion in all operated joints. Mean grip power measured by Jamar dynamometer was 8.4 preoperatively and 8.5 postoperatively. FIG. 9 shows that all patients sensed relieved pain postoperatively: 7 patients were painless (compared to 3 preoperatively) and 4 had only mild pain when using the hand (6 preoperatively). Severe pain sensations occurred only preoperatively. Mean joint space was 2 mm when measured from postoperative x-rays. No synovitis or fistula formations were noticed.

As holds true in all successful MCP arthroplasties, good surgical technique with proper ligament balancing and controlled postoperative rehabilitation are essential for good functional results with these novel biohybrid prostheses. Also, the rehabilitation of the joints must correlate with the biodegradation rate of the bioreplaceable spacer as well as with the tissue ingrowth into the spacer. Thus, occupational therapy needs to be adjusted to be optimal for this novel prosthesis.

While the principles of the invention have been made clear in the illustrative embodiments set forth above, it will be obvious to those skilled in the art to make various modifications to the structure, arrangement, proportion, elements, materials and components used in the practice of the invention. To the extent that these various modifications do not depart from the spirit and scope of the appended claims, they are intended to be encompassed therein.

We claim:

1. A joint prosthesis system for joining a first bone having a first surface to a second bone having a second surface, comprising:
at least one bioabsorbable spacer adapted to be interposed between the first surface and the second surface; and
at least one connector adapted to be fixedly attached to the first bone and the second bone, at least a portion of the connector being in contact with the spacer and disposed to prevent lateral movement of the spacer.

2. The joint prosthesis system as set forth in claim 1, wherein said bioabsorbable spacer is cylindrical.

3. The joint prosthesis system as set forth in claim 1, wherein said bioabsorbable spacer has a porosity of about 50 μm to 1000 μm.

4. The joint prosthesis as set forth in claim 3, wherein said bioabsorbable spacer comprises a bioabsorbable fabric wrapped to form a cylindrical body.

5. The joint prosthesis as set forth in claim 4, wherein said bioabsorbable spacer further comprises a bioabsorbable film that binds with said bioabsorbable fabric.

6. The joint prosthesis as set forth in claim 5, wherein said bioabsorbable film comprises bioactive components.

7. The joint prosthesis system as set forth in claim 4, wherein said bioabsorbable fabric is comprised of at least two compounds having different degradation rates in tissue.

8. The joint prosthesis system as set forth in claim 4, wherein said bioabsorbable fabric is coated with a material having a different degradation rate in tissue than the bioabsorbable fabric.

9. The joint prosthesis system as set forth in claim 7, wherein said bioabsorbable fabric comprises fibers, said fibers comprising a first polymer coated with a second polymer that degrades faster in tissue than said first polymer.

10. The joint prosthesis system as set forth in claim 1, wherein said bioabsorbable spacer comprises a bioabsorbable fabric comprising bioabsorbable fibers having a thickness of about 1 to 300 μm.

11. The joint prosthesis system of claim 1, wherein said bioabsorbable spacer comprises a bioactive agent.

12. The joint prosthesis system as set forth in claim 1, wherein said bioabsorbable spacer comprises a cavity.

13. The joint prosthesis system as set forth in claim 12, wherein the surface of said cavity comprises at least one bioactive agent.

14. The joint prosthesis system as set forth in claim 13, wherein said at least one bioactive agent is a bone growth promoting substance.

15. The joint prosthesis system as set forth in claim 13, wherein said at least one bioactive agent is hyaline cartilage cells.

16. The joint prosthesis system as set forth as set forth in claim 1 comprising two bioabsorbable spacers.

17. The joint prosthesis system as set forth in claim 16, wherein at least one of said bioabsorbable spacers comprises a cavity.

18. The joint prosthesis system as set forth as set forth in claim 17, wherein the surface of said cavity comprises a bioactive agent.

19. The joint prosthesis system as set forth as set forth in claim 17, wherein the surface of said cavity further comprises hyaline cartilage cells.

20. The joint prosthesis system as set forth in claim 1 comprising two bioabsorbable spacers, wherein each of said bioabsorbable spacers comprise a first side adapted to contact either a first bone having a first surface or a second bone having a second surface and each of said absorbable spacers comprise a second side adapted to contact the other bioabsorbable spacer.

21. The joint prosthesis system as set forth in claim 20, wherein said first side comprises a bioactive agent to promote bone growth, and said second side comprises a bioactive agent to promote cartilage growth.

22. The joint prosthesis system as set forth in claim 1, wherein said connector is constructed of the patient's own tissue.

23. A method for treating a joint injury comprising the steps of:
providing at least one bioabsorbable spacer;
interposing said at least one bioabsorbable spacer between the surface of a first bone having a first surface and a second bone having a second surface;
connecting said first bone to said second bone with at least one connector such that at least part of said connector contacts said at least one bioabsorbable spacer, thereby restricting the lateral movement of said bioabsorbable spacer.

24. The method of claim 23, wherein said bioabsorbable spacer is cylindrical.

25. The method of claim 23, wherein said bioabsorbable spacer has a porosity of about 50 μm to 1000 μm.

26. The method of claim 23, wherein said bioabsorbable spacer comprises a bioabsorbable fabric wrapped to form a cylindrical body.

27. The method of claim 26, wherein said bioabsorbable spacer further comprises a bioabsorbable film that binds with said bioabsorbable fabric.

28. The method of claim 27, wherein said bioabsorbable film includes bioactive components.

29. The method of claim 26, wherein said bioabsorbable fabric is comprised of at least two compounds having different degradation rates in tissue.

30. The method of claim 26, wherein said bioabsorbable fabric is coated with a material having a different degradation rate in tissue than the bioabsorbable fabric.

31. The method of claim 29, wherein said bioabsorbable fabric comprises fibers, said fibers comprising a first polymer coated with a second polymer that degrades faster in tissue than said first polymer.

32. The method of claim 23, wherein said bioabsorbable spacer comprises a bioabsorbable fabric comprising bioabsorbable fibers having a thickness of about 1 to 300 μm.

33. The method of claim 23, where said bioabsorbable spacer comprises a cavity.

34. The method of claim 23, wherein two bioabsorbable spacers are interposed between the first and second bones such that the first bioabsorbable spacer is interposed between the first bone and the second bioabsorbable spacer and the second bioabsorbable spacer is interposed between the first bioabsorbable spacer and the second bone.

35. The method of claim 33, wherein at least one of said bioabsorbable spacers comprises a cavity.
